(19) United States
(12) Patent Application Publication
(10) Pub. No.: US 2018/0169062 A1
(43) Pub. Date: Jun. 21, 2018

(54) Palladium Lipoic Acid Complex Formulation As An Adjunct In Radiotherapy

(71) Applicant: Garnett McKeen Laboratory, Bohemia, NY (US)

(72) Inventor: Merrill Garnett, Babylon, NY (US)

(21) Appl. No.: 15/576,939

(22) PCT Filed: May 26, 2016
(86) PCT No.: PCT/US2016/034337

§ 371 Date: Nov. 27, 2017

Related U.S. Application Data
(60) Provisional application No. 62/166,337, filed on May 26, 2015.

(51) Int. Cl. A61K 31/381; A61K 33/24; A61P 35/00; A61K 9/00; A61N 5/10

(52) U.S. Cl. CPC A61K 31/381; A61K 33/24; A61P 35/00; A61N 2005/1098; A61K 9/0019; A61N 5/10; A61K 9/0053

(57) ABSTRACT

The present invention provides a method for enhancing the anti-tumor effects of radiation therapy in a subject in need thereof. The method includes administering an effective amount of a composition comprising a palladium lipoic acid complex prior to, or along with, radiation therapy.

### CROSS REFERENCE OF RELATED APPLICATION

This application is an International application which claims priority to U.S. Provisional Application No. 62/166,337, filed May 26, 2015, which is incorporated by reference herein in its entirety.

### BACKGROUND OF THE INVENTION

The present invention relates generally to the use of a palladium lipoic acid complex as an adjunct in radiotherapy.

Despite the use of chemotherapy and radiotherapy, cancer eradication remains a major challenge to mankind. Radiotherapy, e.g. using external beams such as X-rays, gamma rays, and charged particles to shrink tumors or kill cancer cells, can be used as an adjuvant or neo-adjuvant to surgery and chemotherapy for a majority of cancer patients. For example, radiotherapy is used to treat localized solid tumors, such as cancers of the skin, tongue, larynx, brain, breast, lung, prostate or uterine cervix.

Most of the radiation treatments apply low doses of ionizing radiation (IR) to local regions of the body, which can either damage DNA directly or through free radicals generated within the cells that, in turn, damage the DNA. Radiosurgery/stereotactic body radiation therapy uses high doses of radiation to many cancer types including non-small-cell lung cancer, prostate cancer, renal cell carcinoma and hepatocellular carcinoma with limited toxicity to normal tissue. Similarly, intensity modulated radiation therapy uses non-uniform, computer-optimized radiation fields to deliver a high dose of radiation to the tumor.

Regardless of the type of radiotherapy, radiation exposure causes free radical mediated cytotoxicity to normal cells. This damage to normal, healthy cells must be addressed. Furthermore, this modality alone seldom achieves a satisfactory therapeutic outcome in many cases. Hence, limiting toxicity of radiotherapy without compromising its antitumor efficacy remains a major challenge to clinicians.

There is a long felt need for agents that can enhance the anti-tumor effects of radiation while attenuating the radiation-induced toxicity.

### BRIEF DESCRIPTION OF THE FIGURES

FIG. 1. Effect of Poly-MVA with or without radiation against Ehrlich's ascites carcinoma cell line induced tumor weight Values are mean±SD, n=6, p<0.01 (Dunnett multiple comparison test) significantly different from the control group.

FIG. 2. Effect of Poly-MVA with or without radiation against Dalton's lymphoma ascites cell line induced tumor weight Values are mean±SD, n=6, p<0.01 (Dunnett multiple comparison test) significantly different from the control group.

### SUMMARY OF THE INVENTION

The present invention provides a method for enhancing the anti-tumor effects of radiation therapy in a subject in need thereof. The method comprises administering an effective amount of a composition comprising a palladium lipoic acid complex prior to, or along with, radiation therapy.

In one embodiment of the invention, the subject in need thereof has cancer.

In another embodiment of the invention, the subject in need thereof has Ehrlich's ascites carcinoma (EAC) or Daltons lymphoma ascites (DLA).

According to the present invention, the palladium lipoic acid complex is administered orally to the subject in need. Alternatively, the composition comprising a palladium lipoic acid complex is administered intravenously to the subject in need.

In one embodiment of the invention, the subject in need thereof has a solid tumor, and the composition comprising a palladium lipoic acid complex is administered directly to the solid tumor via injection.

### DETAILED DESCRIPTION OF THE INVENTION

The present invention provides a method for enhancing the anti-tumor effects of radiation therapy in a subject in need thereof. The method comprises administering an effective amount of a composition comprising a palladium lipoic acid complex to the subject.

Palladium lipoic acid complexes have been shown to provide significant antitumor activity, reduce resistance of tumor cells, as well as lower side effects. Applicants have surprisingly discovered that palladium lipoic acid complexes enhance and/or significantly improve anti-tumor effects when given in conjunction with radiation therapy.

One such palladium lipoic acid complex formulation, Poly-MVA (obtained from Garnett McKeen Laboratories, N.Y.), is designed as a liquid crystal polymer with thiamine. It is known that redox polymers are much more efficient at accepting unpaired electrons and donating them, compared to single molecules. Since lipoic acid and thiamine act as co-factors in the conversion of pyruvate to acetyl coA (the first step of aerobic metabolism), this directs palladium lipoic acid complex formulations such as Poly-MVA to the mitochondria. Its ability to provide an electron source during a transient ischemic event and quench reperfusion-induced generation of free radicals demonstrates its role in the aerobic metabolic cascade.

Therefore, a metabolically dysfunctional cancer cell, using glycolysis as its primary energy source, is exploited by a palladium lipoic acid complex formulation such as Poly-MVA. Cancer cells that rely on glycolysis have regressed in their differentiation and should be considered metabolically anaplastic. Palladium lipoic acid complex formulations such as Poly-MVA provide a cellular electron source that can be utilized by radiotherapy.

Thus, it has been discovered that the antitumor activity of palladium lipoic acid complexes are enhanced by the addition of radiation. This is evident from a decreased tumor volume in a radiation plus palladium lipoic acid complex treated group of animals. See Examples below. In addition, no significant DNA damage at 2 Gy radiation has been observed. Hence, it has been concluded that palladium lipoic acid complex fomulations, such as Poly-MVA, show enhanced antitumor activity when administered before or with a mild dose of radiation.

Thus, it has also been discovered that the antitumor activity of radiotherapy is enhanced by the addition of a palladium lipoic acid complex administered prior to, or along with, radiation. This is evident from a decreased tumor volume in a radiation plus palladium lipoic acid complex treated group of animals. See Examples below.

By “enhancing” it is meant that the anti-tumor effects are greatened or increased as compared to the anti-tumor effects seen as a result of administering a palladium lipoic acid complex alone, and/or radiation therapy alone.

Not being bound my theory, it is believed that a synergistic effect is produced when the palladium lipoic acid complex is administered prior to, or along with, radiation therapy. This synergy is seen in the anti-tumor effects of the palladium lipoic acid complex and/or the anti-tumor effects of the radiation therapy.

### Palladium Lipoic Acid Complex

According to the invention, a palladium lipoic acid complex is represented as (palladium)ₘ(lipoic acid)ₙ, wherein m and n are each independently 1 or 2. In a preferred embodiment, both m and n are 1. The bonds of the palladium lipoic acid complex are coordinate covalent. Both of the carboxylic oxygen atoms and the two sulfur atoms of lipoic acid are involved in formation of these coordinate covalent bonds. The lipoic acid in the complex comprises a bent carbon chain with the ends of the chain bonded to the palladium. Crystal studies imply the structure of palladium lipoic acid to be three dimensional with the palladium in the center of the complex.

The lipoic acid moieties of the palladium lipoic acid complex can be either in oxidized or reduced form. Lipoic acid analogues having a shortened or elongated carbon chain can also be used. Lipoic acid derivatives having one to three additional side groups can also be used. The side groups can be attached to one of the sulfur atoms or can be substituted for the hydroxyl group in the carboxyl of the lipoic acid moiety.

The palladium lipoic acid complex of the present invention can further comprise at least one ligand to the palladium lipoic acid complex. The additional ligand can be, for example, acetate, acetylacetonate, amine, bromide, chloride, fluoride, iodide, nitrate, nitrite, oxalate, oxide, pyridine, sulfate and sulfide. The palladium lipoic acid complex can also comprise additional cations such as, for example, sodium, potassium, magnesium, calcium, zinc and tin and anions such as vanadate and molybdate. Other derivatives of lipoic acid known in the art can also be used in the present invention.

Palladium is a transition metal of group VIII of the periodic table. Salts of palladium can also be employed in preparing the palladium lipoic acid complexes of the present invention. Palladium salts can be selected from, for example, palladium acetate, palladium acetylacetonate, palladium ammonium chloride, palladium ammonium nitrate, palladium bromide, palladium chloride, palladium diamine nitrate, palladium diamylamine nitrate, palladium dibromide, palladium difluoride, palladium dioxide, palladium dipyridine nitrite, palladium ethylenediamine nitrite, palladium iodide, palladium monoxide, palladium nitrate, palladium oxalate, palladium oxide, palladium sulfate, palladium sulfide, palladium tetramine dichloride, palladous potassium bromide, palladous potassium chloride, palladous sodium bromide, and palladous sodium chloride. The preferred palladium salts are palladium chloride, palladium bromide, palladium iodide, palladium nitrate, palladium oxide and palladium sulfide.

The palladium lipoic acid complex of the present invention can be produced, for example, by dissolving lipoic acid in a basic solution and adding an acidic solution containing palladium or a salt thereof. The resulting solution is heated to a boil, e.g., to about 100° C., to produce the palladium lipoic acid complex. More specifically, the palladium lipoic acid complex of the present invention can be synthesized, for example, by the following procedure:

(a) adding palladium or a salt thereof to an acidic solution;

(b) heating the palladium-acidic solution to at least about 100° C.;

(c) filtering the palladium-acidic solution from step (b);

(d) dissolving lipoic acid in a basic solution;

(e) adding the dissolved acidic palladium solution from step (c) to the dissolved basic lipoic acid solution from step (d); and

(f) heating the red mixture of lipoic acid and palladium solution to at least about 100° C., for an amount of time sufficient to obtain the dark brown palladium lipoic acid complex.

The palladium or salt thereof is added to the acidic solution in a mole ratio of between about 1 and about 2 moles palladium to between 2 and about 4 moles of acid. Any method for mixing the palladium and acidic solution can be used, for example, stirring or agitation. The palladium-acidic solution can then be heated to a gentle boil, e.g., at least about 100° C., preferably between about 100° and about 200° C., and most preferably at about 100° C.

The acidic solution to which the palladium is added is selected from acids well known in the art. Such acids include perchloric acid, sulfuric acid, hyriodic acid, hydrobromic acid, hydrochloric acid, nitric acid, phosphoric acid, nitrous acid, acetic acid, carbonic acid, and hydrogen sulfide. Preferably, the acidic solution is hydrochloric acid.

The palladium-acidic solution can be filtered by any method generally known in the art. Such methods include, for example, gravity filtration, suction filtration, centrifugation or the like.

In a separate container lipoic acid is added to the basic solution in a molar ratio of between about 1 mole of lipoic acid per 7 moles of base. Any method of mixing the lipoic acid-base solution can be used, for example, stirring or agitation. Any of the above methods of filtration can then be used to eliminate any undissolved residue. The solution is preferably filtered to complete clarity.

The basic solution in which the lipoic acid is dissolved can be selected from bases known in the art, for example, sodium hydroxide, ethanolamine, potassium hydroxide, sodium acetate, dimethylamine, and the like. Preferably, the basic solution is sodium hydroxide.

Next, the dissolved palladium acid solution is added to the lipoic acid basic solution. The initially red mixture of palladium tetra chloride and base-dissolved lipoic acid solution is heated to a gentle boil, e.g., to at least about 100° C., preferably between 100 and 200° C., and most preferably to about 100° C. The solution is generally allowed to boil for about 10 minutes, though this is increased during manufacturing scale-up. The reaction mixture of the palladium and lipoic acid complex converts to a clear dark brown solution. The pH of the palladium lipoic acid complex solution can be adjusted preferably to a pH between about 6 and about 9, and more preferably to a pH of about 7.

Water can be added to the palladium lipoic acid complex solution in an amount sufficient to obtain a concentration of the palladium lipoic acid complex of at least about 0.01M, preferably between about 0.01M and about 0.08M, and most preferably 0.04M.

The palladium lipoic acid complex can also contain trace micronutrients such as for example, antioxidants, molybdenum, rhodium, ruthenium, thiamine, riboflavin, cyanocobalamin, N-acetyl cysteine, and N-formyl methionine. Other antioxidants that can be included in the complex include, for example, selenium, zinc, ascorbic acid, glutathione, lipoic acid, uric acid, carotenes, α-tocopherol, ubiquinol, and melatonin. Carotenoids, vitamins, minerals, flavonoids and other polyphenolic antioxidants can also be added.

### Formulations

A palladium lipoic acid complex formulation and/or a composition comprising a palladium lipoic acid complex of the invention may exist in a solid form or liquid form. Preferably, the formulation and/or composition exists in a liquid form, for example, as a dispersion. More preferably the formulation and/or composition exists in solution.

The formulation and/or composition may include a pharmaceutically acceptable carrier. According to the present invention, a pharmaceutically acceptable carrier is any suitable carrier known to the skilled artisan and will depend upon the dosage form selected. Different routes of administration necessarily require different pharmaceutically acceptable carriers. An identification of such carders may be found in any standard pharmacy text, for example, Remington's Pharmaceutical Sciences, 17th edition, ed. Alfonso R. Gennaro, Mack Publishing Company, Easton, Pa. (1985).

More specifically, examples of pharmaceutically acceptable carriers include pharmaceutical diluents, excipients or carriers suitably selected for the intended route of administration which is consistent with conventional pharmaceutical practice. For instance, for oral administration in the form of tablets or capsules, the active drug components may be combined with any oral non-toxic pharmaceutically acceptable inert carrier such as starch, cellulose, magnesium stearate, dicalcium phosphate, calcium sulfate, mannitol, and the like. Moreover, when desired or necessary, suitable binders, lubricants, disintegrating agents and coloring agents can also be incorporated in the mixture. Suitable binders, for example, include starch, gelatin, natural and synthetic gums such as acacia, sodium alginate, carboxymethylcellulose, polyethylene glycol and waxes. Among the lubricants, there may be mentioned for use in these dosage forms, boric acid, sodium benzoate, sodium acetate, sodium chloride, etc. Disintegrators include, without limitation, starch, methylcellulose, agar, bentonite, guar gum, etc. Flavoring agents and preservatives can also be included where appropriate. In the case of tablets, they can be further coated with the usual coating materials to make, for example, sugar-coated tablets, gelatin film-coated tablets, tablets coated with enteric coatings, tablets coated with films or double-layered and multi-layer tablets.

In a preferred embodiment, the composition consists essentially of a palladium lipoic acid complex and no other active ingredients. Active ingredients are ingredients that directly contribute to the performance of objectives of the composition. For example, an active ingredient in the instant composition would have a direct effect on anti-tumor effects.

### Administration

The composition comprising a palladium-lipoic acid complex may be administered by any enteral or parenteral route.

Parental administration includes intravenous, intramuscular, subcutaneous, intra-dermal, topical, intra-thecal and intra-arterial methods. Enteral administration includes any suitable form for oral consumption including, for example, tablets, pills, liquid gels, capsules, elixir, and troches.

For parenteral administration, for example, the complex must be sterile and pyrogen-free, and are prepared in accordance with accepted pharmaceutical procedures, for example as described in Remington's Pharmaceutical Sciences at pp. 1518-1522. The aqueous sterile injection solutions may further contain anti-oxidants, buffers, bacteriostats, isotonicity adjusters and like additions acceptable for parenteral formulations. Various unit dose and multidose containers, e.g., sealed ampules and vials, may be used, as is well-known in the art. The essential ingredients of the sterile parenteral formulation, e.g., the water and the selected palladium-lipoic acid complex, may be presented in a variety of ways, just so long as the solution ultimately administered to the patient contains the appropriate amounts of the essential ingredients. Thus, for example, the palladium-lipoic acid complex/water formulation may be presented in a unit dose or multidose container, ready for injection. As another example, a concentrated solution of palladium-lipoic acid complex/water may be presented in a separate container from a diluting liquid (water or palladium-lipoic acid complex/water) designed so that the contents can be combined to give a formulation containing appropriate amounts for injection.

As another alternative, the palladium-lipoic acid complex may be provided in a freeze-dried condition in one container, while a separate container contains diluting liquid (water or palladium-lipoic acid complex/water, depending on the amount of palladium-lipoic acid complex in the other container), again designed so that the contents can be combined to give a formulation containing the appropriate amounts of the water and selected palladium-lipoic acid complex. In any event, the contents of each container will be sterile. Suitable carriers for parenteral administration include, for example, water, ethyl alcohol, propylene glycol, ethoxylated isostearyl alcohol, polyoxylated isostearyl alcohol, polyoxyethylene sorbitol and sorbitate esters. In these instances, adequate amounts of sodium chloride, glucose or glycerin can be added to make the preparations isotonic.

### Effective Amount

The dosage of the compositions of the present invention is selected, for example, according to the usage, purpose, conditions and symptoms. Furthermore, the dose administered will be selected, for example, according to the particular composition employed and the size and condition of the patient as well as the route of administration employed, but in any event will be a quantity sufficient to cause a reduction in tumor size.

An effective amount of the palladium lipoic acid complex of the invention is, for example, an amount that results in inhibition of tumor growth and/or reduction in tumor size. For example, when the composition of the present invention is parenterally administered to a patient, a dosage of between about 5 and about 30 ml daily of a 0.04 M solution of the pharmaceutical composition. However, the precise route of administration, dosage and frequency of administration is individualized for each patient and can vary over a wide range depending on the particular disease state being treated, the condition of the patient and the like.

Higher dosages of the palladium-lipoic acid complexes, can be generally administered intravenously, while lower dosages may be given orally or by any injectable route.

### Subject in Need Thereof

A subject in need thereof according to the invention includes any mammal that can benefit from enhancing anti-tumor effects of radiation therapy. This includes any mammal in need of reduction or elimination of a tumor or cancer. For example, suitable mammals include humans, domestic animals such as cats and dogs, and farm animals such as pigs, horses and cows.

In a method for enhancing the anti-tumor effects of radiation therapy, the palladium lipoic acid complex of the invention can be employed as a stand alone therapy, or in conjunction with other treatments such as, for example, other chemotherapeutics.

### Examples

Ehrlich's ascites carcinoma (EAC) and Daltons lymphoma ascites (DLA) cell lines were obtained from Cancer Institute, Adayar, Chennai, India. The cells were maintained in mice by intraperitoneal inoculation of 1×10⁶ viable cells. The palladium lipoic acid formulation was obtained as a gift from Garnett McKeen Laboratory, Inc., USA.

### Irradiation

Irradiation was carried out using a ⁶⁰Co-Theratron Phoenix Teletherapy Unit (Atomic Energy Ltd., Ottawa, Canada) at a dose rate of 1.88 Gy per minute.
Antitumor Effect of Poly-MVA with or without Radiation in Animals Bearing EAC Transplanted Solid Tumor
Albino mice were injected subcutaneously with 1-2×10⁵ live cells of EAC in PBS in the hind limb. After ˜10 days, animals with palpable tumor were divided into 4 groups of 6 animals each. Animals in Group I were kept as untreated control; Group II animals treated with Poly-MVA (2 ml/kg, p.o) once daily for 2 weeks; Group III animals treated with 2 Gy radiations once a week for 2 weeks; Group IV animals treated with Poly-MVA (2 ml/kg, p.o) daily for 2 weeks and 2 Gy whole body gamma radiations from ⁶⁰Co once a week for 2 weeks, 1 hr after the Poly-MVA administration.
Tumor volume was measured once weekly. Animals were sacrificed 24 hrs after the last dose of irradiation/Poly-MVA treatment. The tumor was extirpated, weight measured and percent inhibition was calculated using the formula [C-T]/C where C is the tumor volume or tumor weight of the control group, and T is that of the treated group.
Antitumor Effect of Poly-MVA with or without Radiation in Animals Bearing DLA Transplanted Solid Tumor
Albino mice were injected subcutaneously with 1-2×10⁵ live cells of DLA in PBS in the hind limb. After ˜10 days, animals with palpable tumor size were divided into 4 groups of 6 animals each and treated as described above. Tumor volumes were measured before the treatment, on the 17^{th} day (1 week after the treatment) and again after the commencement of treatment. Animals were sacrificed 24 hrs after the last dose of irradiation/Poly-MVA treatment and EDTA-blood samples were collected. Platelet counts and single cell gel electrophoresis (Comet) assay were performed in the blood to detect the DNA damage. Fifty cells on each slide were selected at random sites for the quantification of the single stranded-DNA breaks using the ‘GASP’ software system. The DNA damage was compared with that of the control group.

### Effect of Poly-MVA Against Radiation Induced DNA Damage

Albino mice were divided into 3 groups of 6 animals each. Group I was kept as an untreated control (normal); Group II animals were treated with a single dose of 4 Gy radiation and kept as a radiation control. Group III animals were treated with Poly-MVA (2 ml/kg, p.o) immediately after the exposure of 4 Gy radiation. The mice were sacrificed 24 hrs after the irradiation and EDTA-blood was collected. Single cell gel electrophoresis (Comet) assay was performed. Fifty cells on each slide were selected at random sites for the quantification of the single stranded-DNA breaks using the ‘GASP’ software system. The DNA damage was compared with that of the control groups.

### Statistical Analysis

All data were represented as mean±SD. The mean values were statistically analyzed using one-way analysis of variance (ANOVA) (using the Graph Pad Instat software package, CA, USA). The significant differences between the groups were further analyzed by Bonferroni's t-test and Dunnett multiple comparison test for treated vs. the control group. A p value of less than 0.05 was considered significant.

### Results

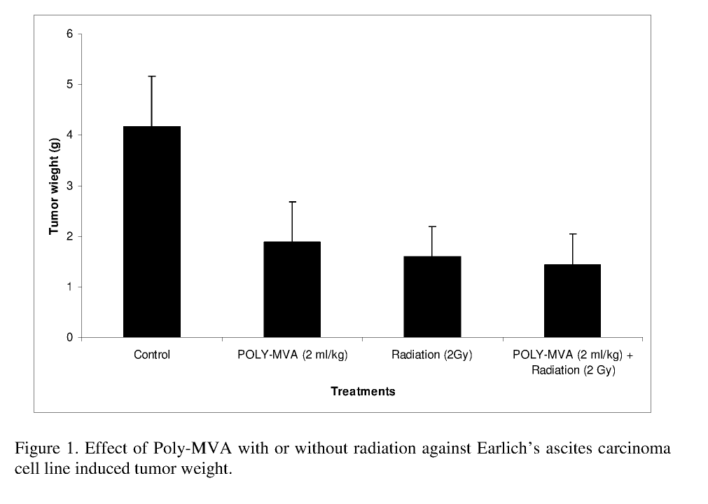

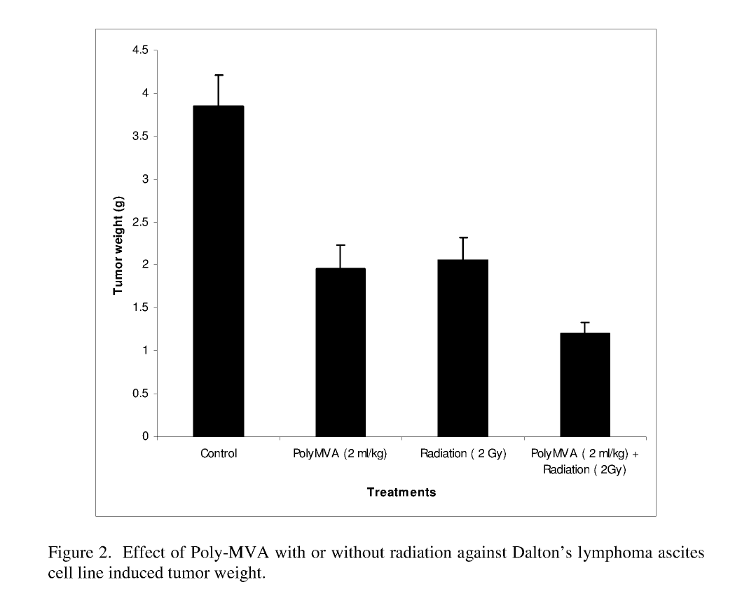

Antitumor Effect of Poly-MVA with or without Radiation Against EAC-Induced Tumor
Radiation alone and administration of Poly-MVA (2 ml/kg, once daily for 2 weeks) in combination with whole body γ-radiation (2 Gy, once per week for 2 weeks) showed significant antitumor effects when compared to the control (tumor bearing animal without treatment) (Table 1). The tumor volumes were 0.38±0.018 cc and 0.37±0.013 cc in the groups treated with radiation alone and Poly-MVA+radiation, respectively. The control group showed tumor volume of 1.02±0.07 cc. Tumor inhibition was highest in the Poly-MVA plus radiation treatment group with regard to tumor weight. While the antitumor effects were found to be significant, with respect to the control group, no statistically significant difference could be observed between the treated groups. Percent inhibition according to the tumor weight was 55, 61 and 65% for the Poly-MVA, radiation alone and Poly-MVA+radiation treated groups respectively (FIG. 1).
Antitumor Effect of Poly-MVA with or without Radiation Against DLA-Induced Tumor
Effect of Poly-MVA on the tumor volume is given in Table 2. Administration of Poly-MVA once daily for 14 days, or radiation alone, could effectively inhibit the tumor growth when compared to that of the control group: 63% and 64% inhibition, respectively. However, the antitumor effect was better for the combination treatment as is evident in the tumor volume of Poly-MVA plus radiation treated group. Tumor weight was significantly reduced in all the treated groups with the maximum decrease observed in the Poly-MVA+radiation treated group (FIG. 2), which demonstrated an 80% inhibition (p<0.001).
No statistically significant effect was observed in the DNA damage in any of the treated groups (Table 3). A slight elevation was observed in the mean value (statistically non-significant) of tail length in the Poly-MVA, radiation alone and also in the Poly-MVA plus radiation treated groups.

TABLE 1
Effect of Poly MVA with or without radiation against the
Ehrlich's ascites carcinoma cell line induced tumor

| | | | | Final tumor |
|---|---|---|---|---|
| | | | Tumor volume | volume |
| | | Tumor volume | 1 weeks after | (2 weeks after |
| | Tumor volume | prior to the | starting the | starting the |
| | prior to | treatment | treatment | treatment) |
| Groups/treatment | treatment | (cm³) | (cm³) | (cm³) |
| Control | — | 0.33 ± 0.05 | 0.64 ± 0.06 | 1.02 ± 0.07 |
| Poly MVA | 2 ml/kg, p.o | 0.31 ± 0.09 | 0.39 ± 0.10* | 0.41 ± 0.01* |
| | | | | (59%) |
| Radiation | 2 Gy | 0.33 ± 0.05 | 0.42 ± 0.13* | 0.39 ± 0.018* |
| | | | | (62%) |
| Poly MVA + | 2 ml/kg, p.o + | 0.32 ± 0.10 | 0.42 ± 0.09* | 0.37 ± 0.013* |
| Radiation | 2 Gy | | | (64%) |

Values are mean ± SD, n = 6 [Value in parenthesis indicate percent inhibition]

TABLE 2
Effect of Poly MVA with or without radiation against the
volume of Dalton's lymphoma ascites induced solid tumor

| | Tumor | | Final tumor volume |
|---|---|---|---|
| | volume | Tumor volume | (2 weeks after |
| | prior to the | 1 weeks after starting | starting the |
| | treatment | the treatment | treatment) |
| Groups | (cm³) | (cm³) | (cm³) |
| Control | 0.44 ± 0.03 | 1.82 ± 0.18 | 2.05 ± 0.31 |
| Poly MVA | 0.44 ± 0.02 | 0.61 ± 0.11** | 0.75 ± 0.21** |
| | | | (63%) |
| Radiation | 0.43 ± 0.02 | 0.67 ± 0.20** | 0.74 ± 0.20** |
| | | | (64%) |
| Poly MVA + | 0.42 ± 0.02 | 0.39 ± 0.17** | 0.42 ± 0.13** |
| Radiation | | | (80%) |

Values are mean ± SD, n = 6
**p < 0.01 (Dunnett multiple comparison test) significantly different from the control group.
[Value in parenthesis indicate percent inhibition]

TABLE 3
Effect of Poly MVA with or without 2 Gy radiation on the
DNA damage in Dalton's lymphoma ascites induced solid tumor

| | | Tail Length | | Olive tail |
|---|---|---|---|---|
| Groups | Tail DNA % | (μm) | Tail moment | moment |
| Control | 0.30 ± 0.21 | 7.36 ± 3.58 | 0.03 ± 0.02 | 0.14 ± 0.08 |
| Poly MVA | 0.44 ± 0.25 | 7.90 ± 2.06 | 0.04 ± 0.02 | 0.15 ± 0.08 |
| Poly MVA + | 0.47 ± 0.19 | 7.84 ± 3.18 | 0.04 ± 0.01 | 0.18 ± 0.09 |
| Radiation | | | | |
| Radiation | 0.45 ± 0.28 | 8.58 ± 3.61 | 0.05 ± 0.03 | 0.19 ± 0.05 |

Values are mean ± SD, n = 6
Values are statistically (Dunnett test) non-significant (p > 0.05) with respect to the control group. P < 0.01 (Dunnett test) significantly different from the control group

What is claimed is:

1. A method for enhancing anti-tumor effects of radiation therapy in a subject in need thereof, wherein the method comprises administering an effective amount of a composition comprising a palladium lipoic acid complex to the subject prior to, or along with, radiation therapy.

2. The method according to claim 1, wherein the subject in need thereof has cancer.

3. The method according to claim 1, wherein the subject in need thereof has Ehrlich's ascites carcinoma (EAC) or Daltons lymphoma ascites (DLA).

4. The method according to claim 1, wherein the composition comprising a palladium lipoic acid complex is administered orally to the subject in need.

5. The method according to claim 1, wherein the composition comprising a palladium lipoic acid complex is administered intravenously to the subject in need.

6. The method according to claim 1, wherein the subject in need thereof has a solid tumor, and wherein the composition comprising a palladium lipoic acid complex is administered directly to the solid tumor via injection.
